(19) United States
(12) Patent Application Publication
(10) Pub. No.: US 2020/0360595 A1
(43) Pub. Date: Nov. 19, 2020

(54) An Injection Device With Needle Cleaning

(71) Applicant: Novo Nordisk A/S, Bagsvaerd (DK)

(72) Inventors: Simon Munch Pedersen, Copenhagen N (DK); Bo Radmer, Hilleroed (DK)

(21) Appl. No.: 16/765,926

(22) PCT Filed: Nov. 19, 2018
(86) PCT No.: PCT/EP2018/081701

§ 371 Date: May 21, 2020

(30) Foreign Application Priority Data
Nov. 21, 2017 (EP) 17202665.0

(51) Int. Cl. A61M 5/00; A61M 5/315; A61M 5/32; A61M 5/24; B08B 9/023

(52) U.S. Cl. CPC A61M 5/001; A61M 5/31585; B08B 9/023; A61M 5/2466; A61M 5/3243

(57) ABSTRACT

The invention relates to a medical injection device having a needle cannula for multiple use and which needle cannula is cleaned between subsequent injections. The needle cannula is preferably cleaned at the distal tip in a cleaning assembly having a hollow cleaning chamber with a variable volume. The distal tip of the needle cannula is maintained inside the cleaning chamber between subsequent injections. The cleaning chamber is defined by an inner surface, a distal seal and a proximal seal. In order to increase the volume of the cleaning chamber during filling of the cleaning chamber one of the seals is formed as a movable plunger which is movable in one direction to expand the volume of the cleaning chamber. To release the movable plunger from the inner surface of the hollow cleaning chamber in order to commence the filling of the cleaning chamber, the movable plunger and the inner wall surface of the hollow cleaning chamber are rotatable in relation to each other during filling of the cleaning chamber.

### THE TECHNICAL FIELD OF THE INVENTION

The present invention relates to an injection device having a cleaning reservoir for cleaning the distal tip of the needle cannula between subsequent injections. The cleaning reservoir is preferably carried by a telescopically movable needle shield covering the needle cannula between injections. In one example, the invention refers to such injection device wherein the cleaning reservoir is filled with the same preservative containing liquid drug as present in cartridge and preferably filled directly from the cartridge.

The present invention also relates to a cleaning assembly for an injection device and more specifically to a cleaning assembly having a cleaning chamber with a variable volume.

### DESCRIPTION OF RELATED ART

A pre-filled disposable injection device for repetitive use is disclosed in European Patent No. 2,911,724 B1. In this prior art document, the same needle cannula is used for multiple injections and the distal tip of the needle cannula is cleaned in a cleaning arrangement between subsequent injections. The cleaning arrangement is exemplified as a hollow reservoir containing a suitable liquid cleaning solvent.

EP 3,062,836 B1 discloses a similar medical injection device which is also intended for multiple injections. The cleaning chamber disclosed therein has a variable volume and is filled with preservative containing liquid drug directly from the cartridge. The active cleaning agent is thus the preservative contained in the liquid drug. Examples of suitable preservatives are phenol and meta-cresol.

Similar injection devices having a cleaning chamber with a variable volume is disclosed in WO2016/162284 and in WO 2017/144601. Both these prior art documents disclose a cleaning chamber which is cylinder-shaped and defined by an inner wall which distally is sealed by a pierceable septum and proximally sealed by a movable plunger. When taking the medical injection device into first use, a volume of the preservative containing liquid drug in the cartridge is transferred to the cleaning chamber by a user initiated filling action. During filling of the cleaning chamber the movable plunger is forced to move axially in the proximal direction until it abuts a stop which thus defines the volume of the cleaning chamber.

The injection devices disclosed in WO2016/162284 and in WO 2017/144601 are thought to be delivered to the user with an empty cleaning chamber which the user has to fill with a cleaning agent in a user operated initiation process. However, after the manufacturer has produced the individual medical injection devices, they are stored and transported often a substantial period of time before arriving at the consumers place. In the relatively long time the injection devices are stored, the movable plunger tend to stick to the inner wall of the cleaning chamber. In order to release the movable plunger from the inner wall of the cleaning chamber, a relatively high force on the movable plunger is needed. Since the force that moves the movable plunger arises solely from the pressure inside the cleaning chamber this again requires a relatively high pressure to be built up in the cleaning agent inside the cleaning chamber during filling of the cleaning chamber.

The resulting force needed to initiate axial movement of the movable plunger inside the cleaning chamber has to overcome a peak value which peak value expresses the stiction between the inner wall of the cleaning chamber and the movable plunger. This stiction depend on the material of the cleaning chamber and of the movable plunger and probably also on the time on storage. In the prior art such cleaning chambers are usually made from a polymer and the movable plungers to slide against the wall surface is often made from natural rubber or a TPE material which often has a relatively high stiction due to the elasticity against the wall surface.

The peak value needed to break a movable plunger loose from a surface to which it sticks is also sometimes referred to as the breakaway force i.e. the force needed to break the movable plunger away from the wall surface.

In the example wherein the cleaning chamber is being filled with preservative containing liquid drug directly from the cartridge, this further requires a relatively high pressure inside the liquid system comprising the cartridge and the lumen of the needle cannula which has the consequence that once the pressure is sufficiently high to release the movable plunger, the movable plunger releases very suddenly once the stiction force is overcome. Once the stiction force is overcome the movable plunger starts to move rapidly axially in the proximally direction due to the resulting force of the relatively high pressure built up in the liquid system which henceforth moves the movable plunger in a rather uncontrollable manner. This leads to over-filling and thus over-pressurizing of the cleaning chamber as the pressure built up inside the liquid system has a resulting force on the movable plunger which is able to move the movable plunger a distance longer than physically possible. Such over-filling and over-pressurizing of the cleaning chamber is highly unwanted as it generates an over-pressure in the entire liquid system resulting in an erroneous dosing.

### DESCRIPTION OF THE INVENTION

It is henceforth an object of the present invention to prevent that the pressure build up inside the cleaning chamber needed to release and break away the movable plunger results in over-pressurization of the cleaning chamber. It is henceforth an object to lower or at least change the force components of the breakaway force to thereby hinder uncontrolled movement of the movable plunger.

Accordingly, in one aspect, the present invention relates to a medical injection device for injection of a liquid drug. This medical injection device comprises:
A housing structure supporting a cartridge containing the liquid drug to be injected,
A needle cannula operationally coupled to the housing structure,
A telescopically movable needle shield covering the distal tip of the needle cannula at least between injections and being rotatable mounted to the housing structure. The needle shield is thus able to both slide telescopically and to rotate in relation to the housing structure. Consequently, the needle shield can perform a helical movement in relation to the housing structure.
A cleaning assembly comprising a chamber part with a hollow cleaning chamber having a variable volume. The variable volume is defined by an inner wall surface of the cleaning chamber, a distal seal and a proximal seal. The cleaning assembly and at least the chamber part thereof is secured to a rotatable needle shield such that at least the chamber part with the cleaning chamber moves rotational with the needle shield in relation to the housing structure. Henceforth, the inner wall surface of the cleaning chamber rotate together with the needle shield.

Further, at least one of the distal seal or proximal seal defining the cleaning chamber comprises a movable plunger which is movable in an axial direction to thereby expand the variable volume of the hollow cleaning chamber.

According to the present invention as defined in claim 1, the movable plunger is inrotatable coupled to the housing structure and as the inner wall surface of the cleaning chamber rotate together with the needle shield, a relative rotation between the needle shield and the housing structure consequently rotate the inner wall surface of the hollow cleaning chamber relatively to the movable plunger.

The relatively rotation occurring between the inner wall of the cleaning chamber and the movable plunger releases the stiction between the chamber wall and the movable plunger in a rotational manner and thus lowers the axial peak force needed to break the two involved parts away from each other.

Accordingly, it is sufficient if the relative rotation only occurs in one rotational direction, namely the rotational direction in which the cleaning chamber is rotated to fill the cleaning chamber. In the opposite direction, the cleaning chamber and the movable plunger could be allowed to rotate together.

Actually, since the two involved elements are rotated in relation to each other, the peak value of the breakaway force needed to break away the movable plunger is overcome in a rotational direction whereby no uncontrolled axial force is required to overcome the peak value of the breakaway force.

Consequently, the two elements are first rotated e.g. helically relative to each other to first overcome the breakaway force where after the two parts are moved away from each other in a controlled axial movement to thereby fully fill the cleaning chamber. The action of overcoming the peak value of the breakaway force is thus separated from the action of filling the cleaning chamber and consequently no pressure or only very little pressure is built up inside the cartridge during the action of overcoming the stiction.

The inner wall of the cleaning chamber is also referred to as the inner surface and is meant to be the circumferential inside of the cleaning chamber which cleaning chamber in one example is made up by a first bore with a relatively large diameter. The bore making up the cleaning chamber is further provided with a channel with a much smaller diameter. Together the first bore and the channel provide a trough going opening in the chamber part. The channel is distally sealed by a distal seal and the larger diameter bore is sealed by the movable plunger which abuts the inner wall of the cleaning chamber. The movable plunger is preferably moved in the proximal direction during filling of the cleaning chamber to thereby expand the volume of the cleaning chamber.

The needle cannula is preferably secured in a needle hub which is guided axially in relation to the housing structure. As a consequence, the needle hub is restricted to only move in the axial direction. During initiation of the injection device, the needle hub is moved proximally in a purely axial movement such that the proximal part of the needle cannula penetrates into the cartridge. In this position, the needle hub locks to the housing structure such that the needle hub cannot move axially nor rotate relatively to the housing structure.

The hub, or in a specific example a hub extension being a part of the hub thus remains in-rotatable in relation to the housing structure and is provided with a guiding structure for guiding the movable plunger. This guiding structure is preferably a track or a groove provided in the hub or alternatively in the hub structure which guides an outwardly pointing protrusion provided on the movable plunger. The movable plunger is thus restricted to only axial movement during rotation of the needle shield.

Alternatively, the guiding structure can be a single track e.g. a ridge such that the movable plunger is only axially guided during one rotational direction. In such case, the movable plunger is only guided axially during filling of the cleaning chamber but is able to rotate together with the cleaning chamber and thus the needle shield in the opposite rotational direction.

In a further example a front element rotationally fixates the cleaning chamber of the cleaning assembly to the telescopically movable needle shield. In this example all three elements; the telescopically movable and rotatable mounted needle shield, the cleaning part with the cleaning chamber and the front element both rotate and move axially in unison.

The movable plunger automatically moves proximally upon filling of the cleaning chamber as the incoming preservative containing liquid drug being pumped from the cartridge and into the cleaning chamber forces the movable plunger to move proximally.

However, in one example one of the cleaning assembly or the movable plunger is provided with a sloped surface engaging the other of the cleaning assembly or the movable plunger. Whenever the cleaning assembly and the movable plunger are rotated in relation to each other this sloped surface will force one of the parts to move axially.

The axial movement of the movable plunger can thus be based either on the increase of pressure in the cleaning chamber or on a mechanical interface, or any combination thereof.

The cleaning assembly is in one example provided with a stop protrusion for engaging the movable plunger to thereby prevent further axial movement of the movable plunger.

The filling of the cleaning chamber is henceforth in one example based on the above mechanical interface between the sloped surface provided on the cleaning assembly and the movable plunger. This mechanical interface defines the minimum filling of the cleaning chamber. However, a certain axial distance can be provided between the minimum filling and the stop protrusion which defines the maximum filling of the cleaning chamber. The difference between the minimum and mandatory filling and the maximum filling is meant to be a buffer zone which is able to obtain various tolerances in the injection device which tolerance can lead to different degrees of filling of the cleaning chamber.

The cleaning chamber can be filled with any cleaning agent suitable of maintaining the distal tip of the needle cannula biological clean between injections. However, in one preferred embodiment, the preservative containing liquid drug in the cartridge is also used as the cleaning agent. In this preferred example, the preservative containing liquid drug is transferrable from the cartridge and into the cleaning chamber by a user initiated initiation process.

Liquid drugs often contain a preservative such as meta-cresol or phenol or the like. In such case the cleaning chamber can be filled with preservative containing liquid drug directly from the cartridge and the volume of preservative containing liquid drug filled into the cleaning chamber works as a cleaning agent due to its content of preservative.

The volume of preservative containing liquid drug required inside the cleaning chamber can be transferred from the cartridge to the cleaning chamber in a number of different ways. One preferred way is to physically move the cartridge in the proximal direction. The cartridge comprises a glass part which distally is sealed by a pierceable septum and a proximal rubber plunger which is movable in the distal direction to thereby decrease the volume of the cartridge. This rubber plunger is usually moved forward by an injection mechanism comprising a piston rod. In normal use such piston rod is prevented from moving proximally and can thus only move distally to decrease the volume of the cartridge. When the cartridge is moved proximally and the piston rod and thus the rubber plunger is prevented from moving in the proximal direction, the pressure inside the cartridge increases and drug is pressed out through the needle cannula inserted through the distal septum.

In one specific example, the needle hub which is moved proximally during the initiation of the injection device is also used to move the cartridge in the proximal direction before the needle hub is clicked to the housing structure.

In a second aspect, the present invention relates to a cleaning assembly for cleaning the distal tip of the needle cannula between injections. Such cleaning assembly which is particular suitable for an injection device of the type referred to the claims 1 to 12, has a hollow cleaning chamber defined by an inner, preferably circumferential, wall surface which is sealed by a distal seal and a proximal seal. Both the distal seal and the proximal seal are preferably configured to be pierced by a needle cannula.

In order to increase the variable volume of the cleaning chamber during filling of the cleaning chamber, one of the distal seal or proximal seal are formed as a movable plunger which radially abuts the inner wall and is movable along the inner wall.

Further, and in order to overcome stiction between the movable plunger and the inner wall, these two parts are rotatable in relation to each other. Preferably by having the movable plunger being guided purely axially while the inner wall surface of the cleaning chamber is rotated. It is thus possible to release and break away the movable plunger from the inner wall of the cleaning chamber by a rotational movement e.g. in combination with an axial movement rather than by a strictly axial movement.

To generate the relative rotation between the cleaning chamber and the movable plunger, the movable plunger is axially guided in the housing structure whereas the cleaning chamber and thus the inner wall surface is coupled to the needle shield to rotate together with the needle shield.

In a further example, the movable plunger is axially guided in the needle hub which is in-rotatable coupled to the housing structure such that the inner wall surface rotate relatively to both the housing structure and to the needle hub.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel but could also be made from a polymeric material or a glass material. The needle cannula can be anchored in a “Needle Hub” or directly to the housing structure of the injection device without the use of a needle hub. If the needle cannula is anchored in a needle hub this needle hub can be either permanently or releasable coupled to the injection device, however in injection devices wherein the same needle cannula is used for multiple injections, the needle hub is usually permanently coupled to the housing structure e.g. by an initiation process which moves the proximal end of the needle cannula into contact with the interior of the cartridge.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. The term “preservative containing liquid drug” is preferably used to describe a liquid drug containing any kind of a preservative. Such liquid drug could in one example be a blood sugar regulating liquid drug such as insulin, insulin analogue, GLP-1 or GLP-2, and the pre-servative contained in the liquid drug could in one example be phenol, meta-cresol or any combination thereof. However any kind of preservative can under this term be combined with any kind of liquid drug.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled injection device” or “Disposable injection device” is meant an injection device containing a predetermined quantum of a liquid drug and which injection device is disposed of once this predetermined quantum has been used. The cartridge containing the liquid drug is permanently positioned or embedded in the injection device such that the user cannot remove the cartridge without permanent destruction of the injection device. Once the predetermined amount of liquid drug in the cartridge and thus in the injection device is used either in one injection or in a series of multiple injections, the user discards the entire injection device including the embedded cartridge.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” or “permanently embedded” as used in this description is intended to mean that the parts, permanently connected or permanently embedded, requires the use of tools in order to be separated and should the parts be separated it would permanently damage at least one of the parts thereby rendering the construction useless for its purpose.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the injection device.

FIG. 2 Show a perspective view of the injection device with the housing structure visually removed.

FIG. 3 show a cross sectional view of the needle shield.

FIG. 4A show a cross sectional view of the distal part of the injection device in FIG. 1 and FIG. 2 with the movable plunger positioned in the most distal position.

FIG. 4B show a cross sectional view of the distal part of the injection device in FIG. 1 and FIG. 2 with the movable plunger positioned in the most proximal position.

FIG. 5 show an exploded view of the distal part of the injection device in FIG. 1 and FIG. 2.

FIG. 6 show a cross sectional view of the cleaning assembly.

FIG. 7 show a side view of the movable plunger.

FIG. 8 show a cross sectional view of the most distal part of the injection device with the movable plunger positioned in the most distal position.

FIG. 9 show a perspective view of the most distal part of the injection device prior to initiation.

FIG. 10 show a perspective view of the most distal part of the injection device during initiation

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “anti (or counter) clockwise” or similar relative expressions are used, these only refer to the appended figures and not to an actual situation of use. The shown figures are schematic representations for which reason the configuration of the different structures as well as their relative dimensions are intended to serve illustrative purposes only.

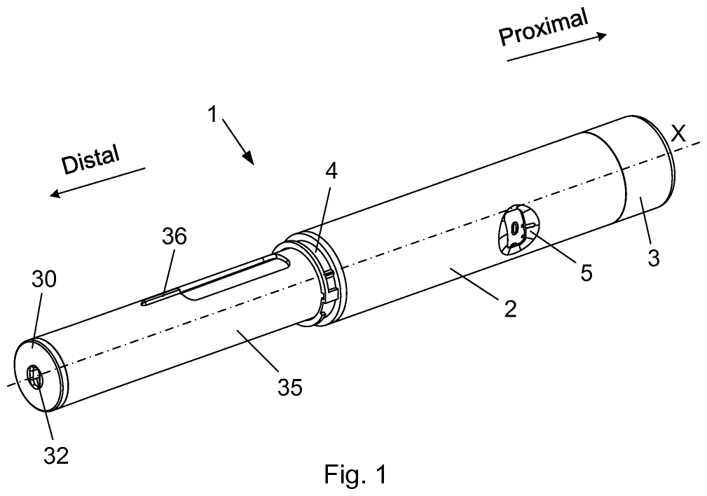

In that context it may be convenient to define that the term “distal end” in the appended figures is meant to refer to the end of the injection device which usually abut or points towards the skin of the user during injection whereas the term “proximal end” is meant to refer to the opposite end pointing away from the skin during injection. The distal end of an injection usually carries the needle cannula whereas the proximal end often is provided with an operational button to set the size of the dose to be injected. Distal and proximal is meant to be along an axial orientation of the injection device along a virtual centre line marked “X” in figure 1.

FIG. 1 discloses a perspective view of the injection device 1 according to the invention. The injection device 1 which in the disclosed embodiment is pen-shaped comprises a housing structure 2 proximally provided with a dose setting button 3 for selecting the size of the individual dose to be injected. The housing structure 1 is provided with a window 5 through which the user can visually inspect a scale drum 70 carrying indicia 71 indicating the size of the dose being set. In the disclosed example on FIG. 1, no dose has been set yet, which is reflected by the indicia “zero” appearing in the window 5. Distally the housing structure 2 holds a telescopically movable needle shield 35 which covers the needle cannula 15 between injections and which telescopically movable needle shield 35 further carries a cleaning assembly 40 for cleaning the distal tip 17 of the needle cannula 15 between injection as will be explained. The telescopically movable needle shield 35 is during injection pressed against the skin of the user and moves proximally against the bias of a not-shown compression spring.

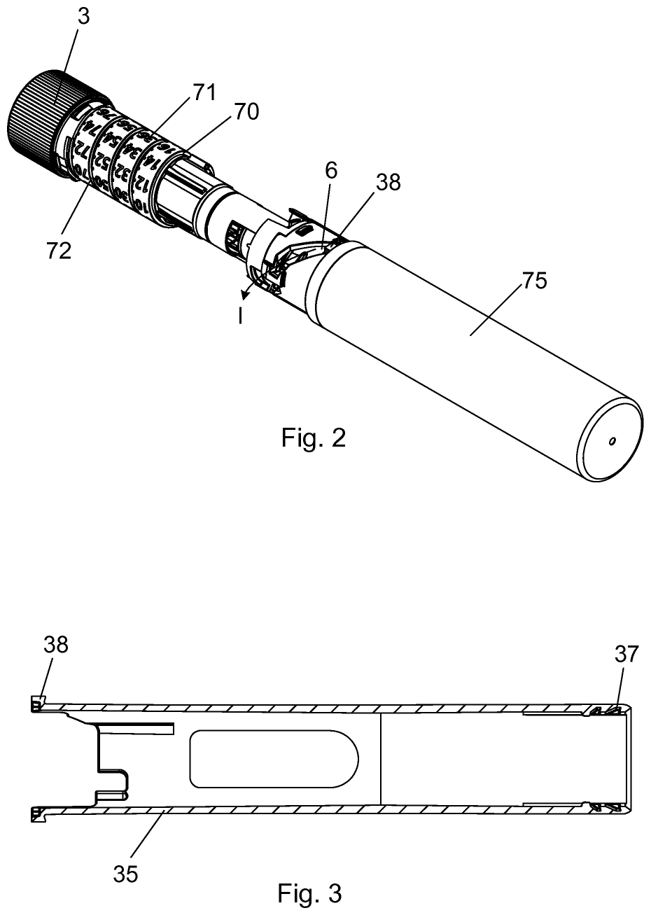

FIG. 2 discloses a further perspective view of the injection device 1 however in FIG. 2 a part of the housing structure 2 has been removed to disclose the inside of the injection device 1. Further, in FIG. 2, the needle shield 35 is covered by a removable protective cap 75 which on its inner surface is provided with a ridge similar to the ridge 36 provided on the outer surface of the needle shield 35 such that a rotation of the protective cap 75 is transferred to a similar rotation of the needle shield 35.

FIG. 2 with the housing structure 2 partly removed also show the scale drum 70 which has a helical track 72 engaging a similar ridge or protrusion on the inner surface of the housing structure 2 such that the scale drum 70 moves helically when rotated. This brings the indicia 71 on the scale drum 70 to pass the window 5 in the housing structure 2 both during dose setting and during dose expelling.

On the inner surface of the protective cap 75 one of more—non-shown—inwardly pointing protrusions are provided. These protrusions engage the peripheral track 4 provided in the housing structure 1 (see e.g. FIG. 1) such that user is required to rotate the protective cap 75 before it can be removed. This rotation is transferred to a similar rotation of the needle shield 35 by engagement with the ridge 36.

The needle shield 35 is further disclosed in FIG. 3. Distally the needle shield 35 is provided with a number of recesses 37 for securing the front element 30 of the cleaning assembly 40 and proximally the needle shield 35 is provided with a pair of outwardly pointing protrusions 38 the use of which will be explained.

In FIG. 2 it is seen that the housing structure 1 is provided with a helical track 6. This helical track 6 is engaged by the outwardly pointing protrusions 38 on the needle shield 35 such that whenever the needle shield 35 is rotated it moves helically. During initiation of the injection device, the user rotates the protective cap 75 in order to remove it. This rotation is trans-ferred to a rotation of the needle shield 35 which thus moves helically in the proximal direction due to the engagement of the protrusions 38 with the helical track 6 as indicated by the arrow “I” in FIG. 2.

Once the outwardly pointing protrusion 38 has been moved through the helical track 6, it enters into an axial track thus allowing the needle shield 35 to telescope axially. The user thus has to unlock the injection device 1 by rotating the needle shield 35 e.g. by way of the protective cap 75 until the outwardly pointing protrusion 38 is aligned in this axial track where after an injection can be performed by pushing the needle shield 35 against the skin of the user. After finalizing the injection and removing the distal end of the needle shield 35 from the skin, a non-shown compression spring urges the needle shield 35 back in distal direction where after the user can lock the injection device 1 by rotating the needle shield 35 such that the outwardly pointing protrusion 38 re-enters the position disclosed in FIG. 2.

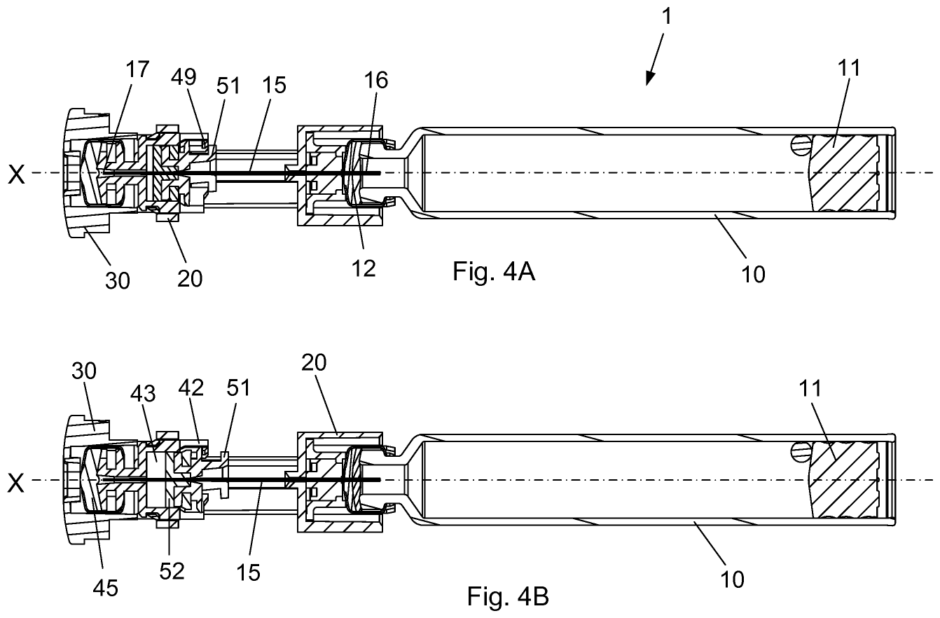

In the following FIGS. 4A to 10) of the distal part of the injection device 1, the telescopically movable needle shield 35 has been visually removed for illustrative purposes alone.

The housing structure 2 holds a cartridge 10 which proximally is provided with a plunger 11 as disclosed in FIG. 4A-B. This plunger 11 is during injection moved forward by a not-shown injection mechanism as it is generally known from injection devices. Distally the cartridge 10 is provided with a septum 12 which is penetrated by the needle cannula 15. When the plunger 11 is moved in the distal direction, the liquid drug contained inside the cartridge 10 is pressed out through the lumen of the needle cannula 15.

The needle cannula 15 has a proximal part 16 which in FIG. 2A-B is penetrated into the cartridge 10. Distally the needle cannula 15 is provided with a sharp tip 17 which is used to penetrate through the skin of user during injection.

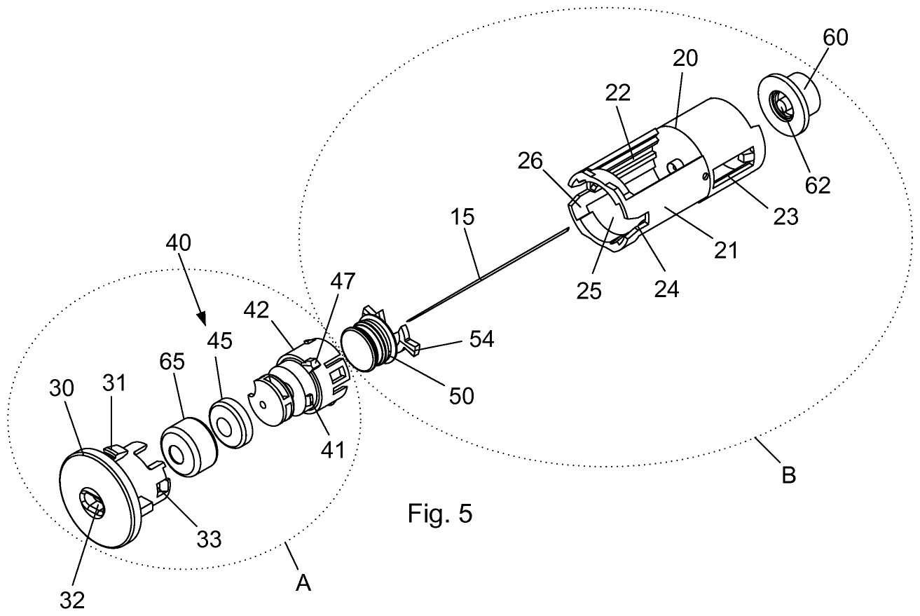

The needle cannula 15 is secured in a hub 20 which as best seen in FIG. 5 has two axial extensions 21 which each on the inner surface is provided with a longitudinal groove 22, the use of which will be explained later. Between these axial extensions 21, an open area 25 is provided. Further, the axial extensions 21 have under-cuts 26 for the passage of outwardly pointing protrusions 47 as will be explained. These under-cuts 26 are provided on both sides of axial guiding tracks 24 as best seen in FIG. 5.

All though the illustrations depict only two extensions 21 any random number of extensions and open areas 25 can be provided.

When the injection device 1 is delivered to the user, the proximal part 16 of the needle cannula 15 has not yet been connected to the cartridge 10 as disclosed in FIG. 8. The actual connection of the proximal part 16 of the needle cannula 15 to the cartridge 10 is done by the user during the initiation of the injection device prior to performing the first injection. Such initiation process is disclosed in details in International application No. PCT/EP2017/065048.

During the initiation, the needle hub 20 is moved axially in relation to the housing structure 2 such that the proximal part 16 of the needle cannula 15 penetrates through the distal septum 12 of the cartridge 10 as disclosed in FIG. 4A-B.

Once the proximal part 16 of the needle cannula 15 has been inserted into the cartridge 10 as disclosed in FIG. 4A-B, a predetermined volume of the liquid drug contained in the cartridge 10 is transferred to the cleaning assembly 40 such that the preservative contained in the liquid drug afterwards are used to clean the distal tip 17 of the needle cannula 15 between injections. This requires that the liquid drug contains a preservative which in one example could be e.g. phenol or meta-cresol.

Distally the telescopically movable needle shield 35 is clicked to a front element 30 which has a number of resilient protrusions 31 engaging the recesses 37 such that the front element 30 and the telescopically movable needle shield 35 move together in unison both axially and rotationally. Alternatively, the front element 30 and the telescopically movable needle shield 35 could be moulded as one structural element. The front element 30 is further provided with a through-going opening 32 through which the distal tip 17 of the needle cannula 15 moves during injection.

At the proximal end the front element 30 is provided with a number of radial openings 33 (FIG. 5) which secures the front element 30 to the cleaning assembly 40 by engaging a number of outwardly pointing protrusions 41 which extend radially from a chamber part 42 of the cleaning assembly 40. The front element 30 and the cleaning assembly 40 thus move together both axially and rotationally.

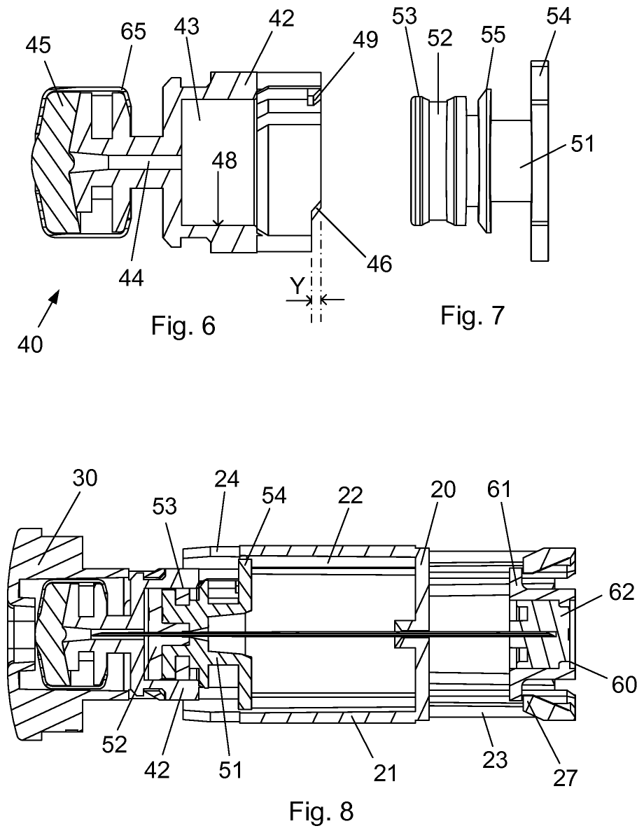

The cleaning assembly 40 which is disclosed in details in FIG. 6 comprises the chamber part 42 which distally is sealed by a front seal 45 connected to the chamber part 42 by a metal bend 65 as commonly known from cartridge production. The chamber part 42 is further provided with an outwardly pointing protrusion 47 (see FIG. 5) the purpose of which will be explained later.

The chamber part 40 is internally divided into a cleaning chamber 43 which proximally is closed by a movable plunger 50 disclosed in FIG. 7. The chamber part 42 is distally provided with a through-going channel 44 which distally is sealed by the front seal 45. The interior of the cleaning chamber 43 and the channel 44 together makes up the volume containing the cleaning solvent for cleaning the distal tip 17 of the needle cannula 15 between injections.

The movable plunger 50 which operates inside the cleaning chamber 43 is disclosed in details in FIG. 7 and is made from a rigid part 51 and one or more flexible parts 52. The rigid part 51 is preferable moulded from a suitable polymer and the flexible part 52 is moulded from a TPE. In a preferred embodiment the two parts 51, 52 are co-moulded in a 2K moulding.

The flexible part 52 has at least one circumferential lip 53 (two in the disclosed embodiment) which seals against the inner surface 48 of the cleaning chamber 43 as e.g. disclosed in FIG. 8. The rigid part 51 has a number of radially extending arms 54 which are guided in the grooves 22 inside the hub extension 21 such that the movable plunger 50 can only slide axially in relation the hub 20 and the hub extension 21.

FIG. 8 discloses the hub 20 and the cleaning assembly 40 with the movable plunger 50 before initiation of the injection device 1. The movable plunger 50 inside the cleaning chamber 43 is positioned at the distal end of this cleaning chamber 43 and the distal tip 16 of the needle cannula 15 is positioned in the channel 44.

The proximal part 16 of the needle cannula 15 is positioned in a movable closing element 60 which comprises an outer rigid part 61 and a more soft inner part 62. The rigid outer part 61 is preferable moulded from a suitable polymer whereas the soft inner part 62 is moulded from a softer TPE. The outer part 61 and the inner part 62 are preferably co-moulded in a 2K moulding.

During the initiation of the injection device 1 prior to performing the first injection, the hub 20 is moved in the proximal direction such that the proximal part 16 of the needle cannula 15 penetrates through the septum 12 of the cartridge 10. During this movement the closing element 60 abuts the cartridge 10 and is thus forced distally by the cartridge 10 as best seen in FIG. 4A-B. The proximal part 16 of the needle cannula 15 is kept sterile when inserted into the flexible part 62 of the closing element 60 but is during initiation moved out of this flexible part 62 and into the cartridge 10 as disclosed in FIG. 4A-B.

FIG. 8 discloses the cleaning assembly 40 and the hub 20 before initiation i.e. with the closing element 60 in its most proximal position. FIG. 8 thus depicts the situation before initiation. FIG. 4A depicts the situation wherein the proximal part 16 of the needle cannula 15 has been penetrated through the septum 12 of the cartridge 10 and preservative containing liquid drug is beginning to be filled into the cleaning chamber 43. FIG. 4B depicts the situation when the cleaning chamber 43 has been filled with preservative containing liquid drug.

The hub 20 is provided with a number of axially extending longitudinal tracks (or grooves) 23 which are guided in the housing structure 2 such that the hub 20 can only move strictly axially. Since the extension 21 of the hub 20 is moulded as a part of the hub 20 this extension 21 also only slides axially. The movement of the hub 20 thus occurs only axially and is preferably generated by a rotation of the movable telescopically movable needle shield 35 as will be explained in the following. At the distal end of the hub extension 21, a further guiding track 24 is provided for guiding the chamber part 42 of the cleaning assembly 40 as will be explained.

When the user rotate the telescopically movable needle shield 35 to initiate the injection device 1, both the front element 30 and the chamber part 42 rotate together with the telescopically movable needle shield 35 due the connections 31, 37; 33, 41 between these elements 35, 30, 42.

Since the hub 20 is only able to move axially due to the tracks 23, the movable plunger 50 which via the arms 54 and the groove 22 in the hub extension 21 are connected to the hub 20 also only move axially.

In FIG. 5, the front element 30 and the cleaning assembly 40 which rotate together with the telescopically movable needle shield 35 and thus relatively to the housing structure 2 are marked “A” and the hub 20, the movable plunger 50 and the closing element 60 that move axially in relation to the housing structure 2 are marked “B”. The rotation of the telescopically movable needle shield 35 thus generates a relative rotation between the flexible part 52 of the movable plunger 50 and the inner surface 48 of the cleaning chamber 43 of the cleaning assembly 40 such that any stiction occurring between the plunger 50 and the inner surface 48 are removed or at least reduced.

In one example, the groove 22 can be formed as a single longitudinal track or ridge such that the relative rotation only occurs in one rotational direction. In such example the parts marked “A” in FIG. 5 thus rotate relatively to the movable plunger 50 in one rotational direction but the parts marked “A” and the plunger 50 rotate together in the opposite rotational direction. The situation in which the relative rotation is created is thus the initiation or filling direction.

The chamber part 42 is further provided with a sloped surface 46 (FIG. 6) which engages with one or more of the arms 54. The result being that when the chamber part 42 rotates and the arm 54 encounters this sloped surface 46, the movable plunger 50 is forced to move axially a distance “Y” as indicated in FIG. 6.

In FIG. 4A, the movable plunger 50 has been moved axially the distance “Y” defined by this sloped surface 46 thus defining the minimum filling of the cleaning chamber 43.

The chamber part 42 is further provided with a stop protrusion 49 which the flange 55 on the movable plunger 50 abuts when the maximum filling is reached as disclosed in FIG. 2B.

Transferring preservative containing liquid drug from the cartridge 10 and into the cleaning chamber 43 of the cleaning assembly 40 is done by creating a relative axial movement between the cartridge 10 itself and the movable plunger 11 positioned inside the cartridge 10. This is preferably done by moving the cartridge 10 in the proximal direction while maintaining the position of the movable plunger 11.

During initiation, the proximal movement of the hub 20 and the closing element 60 are preferably transferred to an axial movement of the cartridge 10 by the closing element 60 pushing the cartridge 10 a short distance in the proximal direction. At the same time the non-shown injection mechanism which includes a piston rod abutting the movable plunger 11 prevents the movable plunger 11 from following the proximal movement of the cartridge 10 which pressurizes the interior of the cartridge 10 and henceforth creates a liquid flow through the lumen of the needle cannula 15 into the cleaning chamber 43.

The hub 20 is for this purpose provided with two inwardly pointing click arms 27 which click fits to the housing structure 2 once the hub 20 has been moved to its final proximal position as depicted in FIG. 10. In this position the hub 20 is prevented from any further movement.

As the preservative liquid drug flows into the cleaning chamber 43 (and the channel 44), the movable plunger 50 is moved in the proximal direction. First the movable plunger 50 is moved proximally by the sloped surface 46 on the chamber part 42. Secondly and depending on the tolerances, the movable plunger 50 is further moveable in the proximally direction by the pressure build up in the cartridge 10. The filling volume that lies between the fixed mechanical filling provided by the sloped surface 46 and the stop protrusion 49 i.e. the volume difference disclosed moving from the FIGS. 4A to FIG. 4B are dedicated as a buffer and is primarily provided to obtain any tolerance variation in the filling procedure.

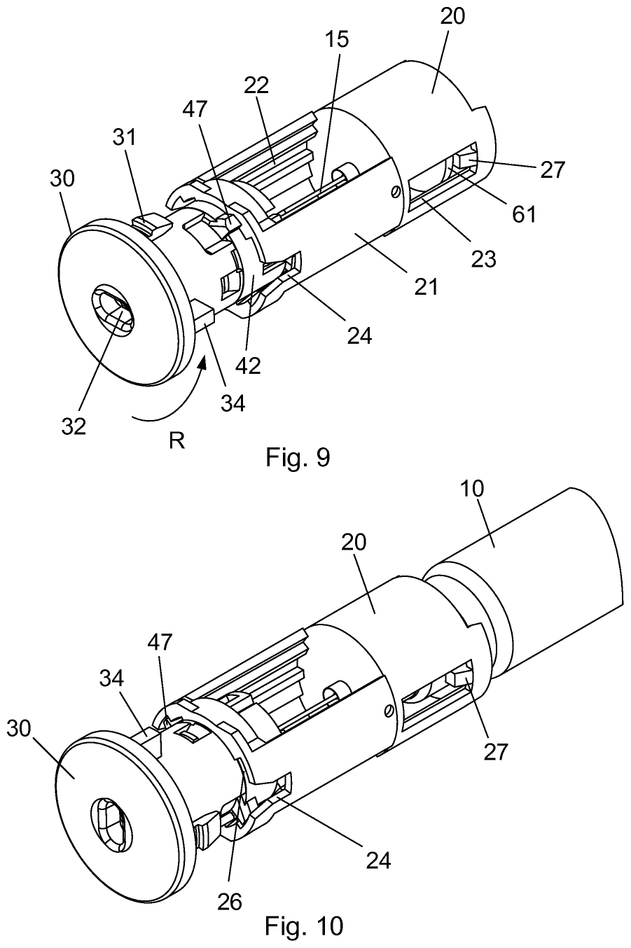

The interaction between the cleaning assembly 40 and the hub 20 is further disclosed in FIG. 9 and FIG. 10. During initiation of the injection device 1, the telescopically movable needle shield 35 is rotated which also rotates the front element 30 and the cleaning assembly 40.

The telescopically movable needle shield 35 is configured such that it moves helically during rotation as disclosed in FIG. 1. As the telescopically movable needle shield 35 moves helically in the proximal direction so does the front element 30 and the cleaning assembly 40.

The helical movement of the cleaning assembly 40 is thus transferred to an axial movement of the hub extension 21 due to engagement between the outwardly pointing protrusion 47 provided on the chamber part 42 and the hub extensions 21. Since the chamber part 42 and thus the outwardly pointing protrusion 47 moves proximally in a helical movement this forces the hub extension 21 and thus the hub 20 to follow the proximal movement in a strictly axial movement along the grooves 23.

FIG. 9 disclose the situation prior to initiation of the injection device 1. The closing element 60 is in the most proximal position as in FIG. 8 and the outwardly pointing protrusion 47 rest against the hub extension 21. FIG. 9 also discloses that the front element 30 is provided with a pair of supporting protrusions 34.

The needle shield 35, the front element 30 and the cleaning assembly 40 is now rotated in the anti-clockwise direction (when viewed from the distal end) as indicated by the arrow “R” in FIG. 7.

FIG. 10 discloses the situation after the front element 30 has been rotated 90 degrees. The supporting protrusions 34 have been rotated away from the alignment with the guiding tracks 24 and the outwardly pointing protrusion 47 has moved into the under-cuts 26 surrounding the axial guiding track 24.

In the 90 degree rotation from the position in FIG. 9 to the position in FIG. 10, the hub 20 is moved axially a distance sufficient to move the proximal part 16 of the needle cannula 15 through the septum 12 of the cartridge 10. At the same time the closing element 60 pushes the cartridge 10 proximally a distance such that a volume of preservative containing liquid drug is transferred from the cartridge 10 and into the cleaning chamber 43. The forces moving the cartridge 10 in the proximal direction are actually transferred from the hub 20 to the cartridge 10 by the rigid part 61 of the closing element 60.

After the first 90 degrees rotation (from FIG. 9 to FIG. 10), the outwardly pointing protrusion 47 is positioned in distal end of the under-cuts 26.

Also, in the position disclosed in FIG. 10, the click arms 27 has clicked into engagement with the housing structure 2 such that the hub 10 is prevented from further movement.

A further rotation of the needle shield 35 and the front element 30 by an additional 90 degrees forces the outwardly pointing protrusion 47 to move through the under-cuts 26 in the inner surface of the hub 20 such that the outwardly pointing protrusion 47 enters into the open area 25. In this position, with the outwardly pointing protrusion 47 positioned in the open area 25, the needle shield 35 and the cleaning assembly 40 can be moved axially in relation to the hub 20 as the needle shield 35 is pressed against the skin of user during injection. This is possible since in this position (180° rotation of the needle shield 35) has moved the outwardly pointing protrusions 38 into the axial track connected to the helical track 6 (see e.g. FIG. 2). During injection the supporting protrusions 34 slides in the guiding tracks 24 thus guiding the cleaning assembly 40 axially.

Some preferred embodiments have been disclosed in the foregoing, but it should be stressed that the invention is not limited to these, but may be embodied in other ways within the subject matter defined in the following claims.

1. A medical injection device for injection of a liquid drug, comprising:
a housing structure supporting a cartridge containing the liquid drug,
a needle cannula mounted relatively to the housing structure such that a distal tip extend in a distal direction and a proximal part extend in a proximal direction and connects with the cartridge at least during injection,
a cleaning assembly comprising a chamber part with a hollow cleaning chamber having a variable volume and defined by an inner surface, a distal seal and a proximal seal, wherein
one of the distal seal or proximal seal comprises a movable plunger which is movable in one direction to thereby expand the variable volume of the hollow cleaning chamber and wherein the movable plunger and the inner wall surface of the hollow cleaning chamber are rotatable in relation to other.

2. A medical injection device for injection of a liquid drug according to claim 1, wherein the cleaning assembly is secured to an telescopically movable needle shield.

3. A medical injection device for injection of a liquid drug according to claim 2, wherein the telescopically movable needle shield and the housing structure are rotatable in relation to each other.

4. A medical injection device for injection of a liquid drug according to claim 3, wherein the inner wall of the cleaning assembly rotates together with the telescopically movable needle shield.

5. A medical injection device for injection of a liquid drug according to claim 2, wherein a front element rotationally fixates the cleaning assembly to the telescopically movable needle shield

6. A medical injection device for injection of a liquid drug according to claim 1, wherein the movable plunger is guided to move axially in relation to the housing structure.

7. A medical injection device for injection of a liquid drug according to claim 1, wherein a hub carrying the needle cannula is guided axially in relation to the housing structure.

8. A medical injection device for injection of a liquid drug according to claim 7, wherein the movable plunger is provided with an outwardly pointing protrusion which is guided in a track or groove provided in the hub or in a hub extension connected to the hub.

9. A medical injection device for injection of a liquid drug according to claim 1, wherein one of the cleaning assembly or the movable plunger is provided with a sloped surface engaging the other of the cleaning assembly or the movable plunger to thereby introduce an axially movement upon relative rotation between the cleaning assembly and the movable plunger.

10. A medical injection device for injection of a liquid drug according to claim 1, wherein the cleaning assembly is provided with a stop protrusion for engaging the movable plunger to thereby prevent further axial movement of the movable plunger.

11. A medical injection device for injection of a liquid drug according to claim 1, wherein the cartridge and the cleaning chamber contains the same preservative containing liquid drug.

12. A medical injection device for injection of a liquid drug according to claim 11, wherein the preservative containing liquid drug is transferrable from the cartridge to the cleaning chamber.

13. A medical injection device for injection of a liquid drug according to claim 12, wherein the cartridge is movable in the proximal direction in relation to the housing structure.

14. A medical injection device for injection of a liquid drug according to claim 13, wherein the cartridge is forced in the proximal direction by the hub.

15. A cleaning assembly for an injection device, comprising;
a hollow cleaning chamber defined by an inner wall sealed by a distal seal and a proximal seal, wherein,
the distal seal and the proximal seal are pierceable preferably by a needle cannula, and
one of the distal seal or proximal seal is a movable plunger which radially abuts the inner wall and is movable along the inner wall, and
wherein, the movable plunger and the inner wall are rotatable in relation to each other.
